(19) United States
(12) Patent Application Publication
(10) Pub. No.: US 2005/0013810 A1
(43) Pub. Date: Jan. 20, 2005

(54) Regulating immune response using dendritic cells

(76) Inventors: Edmund K Waller, Atlanta, GA (US); Hilary S Rosenthal, Lilburn, GA (US); Sagar Lonial, Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 10/477,012

(22) PCT Filed: May 8, 2002
(86) PCT No.: PCT/US02/14497

Related U.S. Application Data
(60) Provisional application No. 60/289,625, filed on May 8, 2001.

(51) Int. Cl. A61K 39/395

(52) U.S. Cl. 424/144.1; 424/93.71

(57) ABSTRACT

Disclosed within a method of altering immune responses using dendritic cells. One form of the method is a method of inducing immunological tolerance in an individual, where type 2 dendritic cells are administrated to an individual, and where the dendritic cells have been incubated with one or more antigens. Another form of the method involves altering an immune response, in which liposomes containing where liposomes containing one or more antigens are administrated to an individual, and where the liposomes are modified with surface-bound molecules that target the liposomes to type 2 dendritic cells. Another form of the method involves reducing immune responsiveness, where liposomes containing one or more antigens are administrated to an individual and where the liposomes are modified with the surface bound molecules that target the liposomes to type 1 dendritic cells or type 2 dendritic cells. Another form of the method is a method of enhancing immune responsiveness, where liposomes containing one or more antigens are administrated to an individual, and where the liposomes are modified with surface-bound molecules that target the liposomes to mature type 1 dendritic cells.

### CROSS-REFERENCE TO RELATED APPLICATIONS

This application claims benefit of U.S. Provisional Application No. 60/289,625, filed May 8, 2001, which application is hereby incorporated herein by reference in its entirety.

### FIELD OF THE INVENTION

The disclosed invention is generally in the field of altering immune responses and specifically in the field of altering immune responses via dendritic cells.

### BACKGROUND OF THE INVENTION

Dendritic cells have specialized capacity to present peptide antigens to T-cells and regulate the initiation of the immune response (Hart, D. N. (1997) “Dendritic cells: unique leukocyte populations which control the primary immune response.” Blood 90: 3245-3287; Bancereau, J. and R. M. Steinman (1998) “Dendritic cells and the control of immunity.” Nature 392: 245-252). There is significant heterogeneity within the dendritic cell lineage. Dendritic cells develop from hematopoietic progenitor “stem” cells under the influence of cytokines which act during early myelo-monocytic differentiation (Peters, J. H., J. Ruppert, et al. (1991) “Differentiation of human monocytes into CD14 negative accessory cells: do dendritic cells derive from the monocytic lineage?” Pathobiology 59: 122-126;Santiago, S. F., E. Belilos, et al. (1992) “TNF in combination with GM-CSF enhances the differentiation of neonatal cord blood stem cells into dendritic cells and macrophages.” J Leukoc Biol 52(3): 274-81; Grouard, G., M. C. Rissoan, et al. (1997) “The enigmatic plasmacytoid T cells develop into dendritic cells with interleukin (IL)-3 and CD40-ligand.” J Exp Med 185: 1101-11; Olweus, J., A. BitMansour, et al. (1997) “Dendritic cell ontogeny: a human dendritic cell lineage of myeloid origin.” Proc Natl Acad Sci USA 94: 12551-6; Rissoan, M. C., V. Soumelis, et al. (1999) “Reciprocal control of T helper cell and dendritic cell differentiation.” Science 283: 1183-6; Siegal, F. P., N. Kadowaki, et al. (1999) “The nature of the principal type 1 interferon-producing cells in human blood.” Science 284: 1835-7; Bergthiier, R., C. Martinon-Ego, et al. (2000) “A two step-culture method starting with early growth factors permits enhanced production of functional dendritic cells from murine splenocytes.” Immunological Methods 239: 95-107; Ferlazzo, G., J. Klein, et al. (2000) “Dendritic cells generated from CD34+ progenitor cells with flt3 ligand, c-kit ligand, GM-CSF, IL4, and TNF-alpha are functional antigen-presenting cells resembling monocyte-derived dendritic cells.” Journal of Immunotherapy 23: 48-58). Phenotypic and functional analyses have demonstrated two general types of dendritic cells, type 1 (DC 1) and type 2 (DC2), that differ in surface marker expression as well as their functional effect on cognate T-cells. Studies of cytokines used to mobilize peripheral blood hematopoietic progenitors have indicated increased numbers of DC I after administration of Flt3 and GM-CSF, while increased numbers of DC2 have been noted after treatment with G-CSF (Arpinati, M., C. L. Green, et al. (2000) “Granulocyte-colony stimulating factor mobilizes T helper-2-inducing dendritic cells.” Blood 95: 2484-2490). Interaction of DC1 with T-cells results in polarization of the T-cells towards Th1 and Tc 1 immune responses characterized by the generation of cytotoxic effectors and production of IL12, TNF and IFN-gamma. In contrast, interaction of DC2 with T-cells leads to T-cells that facilitate humoral immune responses, secrete IL4 and IL4, and suppress cytotoxic Th1 immune responses.

DC have an important role in the anti-tumor effect of autologous and allogeneic transplantation. Antigen primed DC in have been used with promising results in adoptive vaccination against tumors (Mayordomo, J. I., T. Zorina, et al. (1995) “Bone marrow-derived dendritic cells pulsed with synthetic tumour peptides elicit protective and therapeutic antitumour immunity.” Nature Medicine 1(12): 1297-302; Reichardt, V. L., C. Y. Okada, et al. (1999) “Idiotype vaccination using dendritic cells after autologous peripheral blood stem cell transplantation for multiple myeloma—a feasibility study.” Blood 93(7): 2411-9). DC1 promote Th1 immune responses in responding CD4⁺ T-cells characterized by enhanced INF-gamma, TNF, and IL-12 synthesis. DC2 promote Th2 responses in CD4+ T-cells characterized by IL4 and IL10 synthesis, and inhibition of INF-gamma and TNF production in cognate T-cells (Rissoan, M. C., V. Soumelis, et al. (1999) “Reciprocal control of T helper cell and dendritic cell differentiation.” Science 283: 1183-6). Bone marrow contains monocytes and CD86⁺, CD34⁺ progenitor cells which can differentiate into DC1 in the presence of TNF and GM-CSF (Sallusto, F. and A. Lanzavecchia (1994) “Efficient presentation of soluble antigen by cultured human dendritic cells is maintained by granulocyte/macrophage colony-stimulating factor plus interleukin 4 and downregulated by tumor necrosis factor a.” Journal of Experimental Medicine 179: 1109-1118; Ye, Z., A. P. Gee, et al. (1996) “In vitro expansion and characterization of dendritic cells derived from human bone marrow CD34+ cells.” Bone Marrow Transplantation 18(5): 997-1008; Ryncarz, R. E. and C. Anasetti (1998) “Expression of CD86 on human marrow CD34(+) cells identifies immunocompetent committed precursors of macrophages and dendritic cells.” Blood 91(10): 3892-900), as well as CD123^{bright}DC progenitors which differentiate into DC2 in the presence of IL3 (Olweus, J., A. BitMansour, et al. (1997) “Dendritic cell ontogeny: a human dendritic cell lineage of myeloid origin.” Proc Natl Acad Sci U S A 94: 12551-6). Most studies involving hematopoietic progenitor cell transplantation to date have examined the role of DC1 as a method of adoptive immunotherapy for cancer following autologous HPC transplantation (Mayordomo, J. I., T. Zorina, et al. (1995) “Bone marrow-derived dendritic cells pulsed with synthetic tumour peptides elicit protective and therapeutic antitumour immunity.” Nature Medicine 1(12): 1297-302). One recent report, using a murine transplant model, indicated that only host CD11c+DC (DC 1) were necessary for the development of acute GvHD (Shlomchik, W. D., M. S. Couzens, et al. (1999) “Prevention of graft versus host disease by inactivation of host antigen-presenting cells.” Science 285: 412-5). Donor DC did not appear to efficiently present host antigens via cross-priming in a way that led to the initiation of GvHD (Shlomchik, W. D., M. S. Couzens, et al. (1999) “Prevention of graft versus host disease by inactivation of host antigen-presenting cells.” Science 285: 412-5). In this study, the possible effect of donor DC on inhibiting GvHD was not examined. The potential role of increased numbers of donor DC2 in regulating GvHD after G-CSF mobilized allogeneic peripheral stem cell transplants has recently been recognized (Arpinati, M., C. L. Green, et al. (2000). “Granulocyte-colony stimulating factor mobilizes T helper-2-inducing dendritic cells.”Blood 95: 2484-2490).

### BRIEF SUMMARY OF THE INVENTION

In accordance with the purposes of this invention, as embodied and broadly described herein, this invention, in one aspect, relates to a method of inducing immunological tolerance in an individual, where the method comprises administering to the individual type 2 dendritic cells or immature type 1 dendritic cells, where the dendritic cells have been incubated with one or more antigens. In another aspect, the invention relates to a method of reducing immune responsiveness to particular antigens, where the method comprises administering to the individual liposomes, where the liposomes contain one or more antigens, and where the liposomes are modified with surface-bound molecules that target the liposomes to either type 2 dendritic cells or mature type 1 dendritic cells. In another aspect, the invention relates to a method of enhancing immune responsiveness, where the method comprises administering to the individual liposomes, where the liposomes contain one or more antigens, where the liposomes are modified with surface-bound molecules that target the liposomes to mature type I dendritic cells.

In these various aspects, the antigens can be, for example, carbohydrates, nucleic acids, peptides, lipids, or a combination of two or more of carbohydrates, nucleic acids, peptides, and lipids. In particular, the antigens can be MHC, QA, HIV gag, pol, and env (DNA or protein), rheumatoid factor, ICA 89, peripherin, carboxypeptidase H, glutamic acid decarboxylase (GAD), AHNAK, mylein basic protein, retinal S antigen, galactomannoprotein, neuraminidase, influenza matrix protein M1, CMV env proteins pp60, ds DNA, thyroglobulin, insulin, pancreatic islet beta cell antigens, MART-1 (melanoma protein), tyrosinase-related protein 2 (TRP2), melanoma cell lysate, MAGE-A3, allogeneic cell lysate, HLA antigens (protein or DNA), CD31 (protein or DNA), aspergillus chitin, pancreatic carcinoma cell line Panc-1 lysate, ErbB-2/neu, human epithelial cell mucin (Muc-1), Ha-ras oncogene product, p53, carcino-embryonic antigen (CEA), raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), prostate specific membrane antigen (PSMA), HPV-16, MUM, alpha-fetoprotein (AFP), CO17-1A, GA733, gp72, p53, ras oncogene product, HPV E7, Wilm's tumor antigen-1, telomerase, melanoma gangliosides, malignant B cell antigen receptor, malignant B cell immunoglobulin idiotype, variable region of an immunoglobulin, hypervariable region or complementarity determining region (CDR) of a variable region of an immunoglobulin, malignant T cell receptor (TCR), variable region of a TCR, hypervariable region of a TCR, or a combination.

The antigens also can be auto-immune antigens, such as antigens involved in diabetes mellitus, multiple sclerosis, Chron's disease, inflammatory bowel disease, rheumatoid arthritis, thyroiditis, vitiligo, and/or systemic lupus erythematosis. The antigens can be antigens involved in allograft rejection, such that the induced tolerance reduces the allograft rejection. The antigens also can be antigens involved in graft rejection of hematopoietic stem cell transplants, such that the induced tolerance reduces the graft rejection. The antigens also can be antigens involved in graft verses host disease.

For those aspects of the disclosed method involving induction of immunological tolerance, the immunological tolerance can reduce an auto-immune response, such as an auto-immune response involved in diabetes mellitus, multiple sclerosis, Chron's disease, inflammatory bowel disease, rheumatoid arthritis, thyroiditis, vitiligo, and/or systemic lupus erythematosis. The immunological tolerance can reduce an allergic immune response, such as an allergic immune response is involved in asthma, gluten allergy, and atopic dermatitis. The graft rejection immune response can comprise graft rejection and/or complications associated with graft rejection. The immunological tolerance can reduce graft verses host immune response or graft rejection immune response. The individual can be an allograft recipient, such as a hematopoietic stem cell recipient.

The disclosed method of administration of antigen incubated dendritic cells can have additional features and aspects. For example, the incubation step can occur or be performed ex vivo, and type 2 dendritic cells can be isolated prior to the incubation by selection with an anti-BDCA-2 antibody. In the case of type 2 dendritic cells, the dendritic cells can be further purified selection for CD123+ cells and/or removal of CD11c− cells. Thus, in one aspect of the disclosed method, the type 2 dendritic cells are isolated prior to the incubation with antigen by selection with an anti-BDCA-2 antibody, selection for CD 123+ cells, and removal of CD11c− cells. Alternatively, type 1 dendritic cells can be isolated with an anti-BDCA-1 antibody. Type 1 dendritic cells can be further purified by removal of BDCA-2+ and/or CD123+ cells.

The disclosed method of administration of liposomes targeted to dendritic cells can have additional features and aspects. For example, the surface-bound molecule can be a monoclonal antibody, or a peptide high-affinity ligand. In some embodiments, the surface-bound molecules can be specific for CD11c+, BDCA-1, or both. This targets mature type 1 dendritic cells In other embodiments, the surface-bound molecules can be specific for CD123, BDCA-2, BDCA-4, or a combination. This targets type 2 dendritic cells. The disclosed method can have a variety of effects and objects. For example, the liposomes can enhance anti-tumor immune responses, can enhance anti-cancer immune responses, can reduce an auto-immune response. In the latter case, the auto-immune response can be involved in diabetes mellitus, multiple sclerosis, Chron's disease, inflammatory bowel disease, rheumatoid arthritis, thyroiditis, vitiligo, and/or systemic lupus erythematosis. The liposomes can reduce an allergic immune response, such as an allergic immune response is involved in asthma, gluten allergy, and atopic dermatitis. The liposomes can reduce graft verses host immune responses in individuals who are hematopoietic allograft recipients, such as hematopoietic stem cell transplant recipients. The liposomes can reduce graft rejection immune responses such as those that are complications associated with organ allograft transplants.

In another aspect, the invention relates to a method of modifying liposomes, where the method comprises packaging one or more antigens into a lipopsome, where the liposome is modified with surface-bound molecules that target the liposome to either type 2 dendritic cells or immature type 1 dendritic cells.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 is a graph of different cells and treatment versus counts per minute (cpm; representing proliferation of the cells). As described in the Example, 50,000 DC were cultured with 50,000 T-cells in the presence of allo-antigen or 1 μg/ml Con A.

FIG. 2 is a graph of responder to stimulator ratio versus counts per minute (cpm; representing cell proliferation). As described in the Example, serial dilutions of purified DC were prepared with 50,000, 25,000, 12,500, 6,250, 3125, 1500, or 75 cells added to culture wells in combination with 50,000 T-cells.

### DETAILED DESCRIPTION OF THE INVENTION

The present invention may be understood more readily by reference to the following detailed description of preferred embodiments of the invention and the Example included therein and to the Figures and their previous and following description.

It is to be understood that this invention is not limited to specific synthetic methods, specific preparative and analytical techniques unless otherwise specified, or to particular reagents unless otherwise specified, as such may, of course, vary. It is also to be understood that the terminology used herein is for the purpose of describing particular embodiments only and is not intended to be limiting.

Immature DC may be immuno-suppressive, and decrease antigen specific immune responses. A recently published study in normal donors evaluated the ability of immature dendritic cells to augment T-cell activation after in vitro exposure to antigen (Dhodapkar, M. V., C. Y. Li, et al. (1994) “Clinical spectrum of clonal proliferations of T-large granular lymphocytes: a T-cell clonopathy of undetermined significance?” Blood 84(5): 1620-7). Immature dendritic cells were derived from peripheral blood monocytes and then exposed to the influenza peptide MP, and KLH. After return of these DCs to the host, a marked decline in the frequency of MP specific INF-gamma producing cells was noted, and persisted for 30 days. At the same time, the frequency of influenza specific T-cells was unchanged suggesting that the decrease in MP specific INF producing cells was related to DC exposure to the antigen, rather than a global reduction in influenza response. Also, this reduction in MP specific INF-gamma producing cells was paralleled by an increase in the frequency of IL-10 producing MP specific cells, and a reduction in the ability of CD8+ Mp specific cells to kill target cells that had been loaded with Mp peptides. There was no reduction in the overall frequency of MP specific T-cells after return of the immature DC's to account for this change in antigen response.

Recently, a new paradigm has been developed in allogeneic hematopoietic cell transplantation, namely, that donor DC2 inhibit chronic GvHD and GvT responses among recipients of allogeneic HPC transplants. Based upon the naturally occurring biologic variation of the DC2 content in bone marrow grafts from 113 normal allogeneic sibling donors, it was found that recipients of larger numbers of DC2 experienced more relapse, while recipients of fewer DC2 had less relapse and more chronic GvHD post-transplant (Waller, E. K., H. Rosenthal, et al. (2001) “Larger numbers of CD4(bright) dendritic cells in donor bone marrow are associated with increased relapse after allogeneic bone marrow transplantation.” Blood 97(10): 2948-56). Patients received un-manipulated bone marrow from HLA-matched siblings for the treatment of lymphoma, leukemia, and multiple myeloma. The quantity of CD3+ and CD34+ cells and DC2 content in the bone marrow grafts were measured by flow cytometry and correlated with clinical outcomes post-transplant. When the study was initiated, a rare population of CD3−, CD4^{bright}, CD8−, low side scatter, bone marrow cells was noted. These cells were clearly distinct from CD4+ T-cells and CD4^{lo} monocytes. The CD3−, CD4^{bright} are identical to DC2 (CD123+, CD11c−) subsequently described in peripheral blood, lymph node, thymus, and bone marrow (Grouard, G., M. C. Rissoan, et al. (1997) “The enigmatic plasmacytoid T cells develop into dendritic cells with interleukin (IL)-3 and CD40-ligand.” J Exp Med 185: 1101-11; Olweus, J., A. BitMansour, et al. (1997) “Dendritic cell ontogeny: a human dendritic cell lineage of myeloid origin.” Proc Natl Acad Sci USA 94: 12551-6). For these and other reasons, it was realized that dendritic cells could be used to alter immune responses using the disclosed method. Further, it was realized that functional differences between DC1 and DC2 can be exploited using the disclosed method.

A. Definitions

As used in the specification and the appended claims, the singular forms “a,” “an” and “the” include plural referents unless the context clearly dictates otherwise. Thus, for example, reference to “a liposome” includes mixtures of two or more such liposomes, and the like.

Ranges may be expressed herein as from “about” one particular value, and/or to “about” another particular value. When such a range is expressed, another embodimentincludes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Dendritic cell” or “DC”, as used herein, refers to a mature antigen presenting cell,which is identified by the expression of one or more of the following markers on its cell surface: CD40, CD80, CD86, CD83 and HLA-DR, or a dendritic cell progenitor, as defined below, or both.

“Dendritic cell progenitor”, “dendritic progenitor cells”, “dendritic precursor cell”, “dendritic cell precursor”, “DCP”, or “immature dendritic cell”, used interchangeably herein, means a hematopoietic cell that differentiates intro a mature DC. Type 1 Dendritic cell progenitors are typically identified by the expression of one or more of the following markers on its cell surface: CD11c, CD13, CD14, CD33, CD34, HLA_DR, or CD4. Type 2 Dendritic cell progenitors are typically identified by the expression of one or more of the following markers on its cell surface: CD123, CD45RA, CD34, CD36, BDCA-2, BDCA-4, HLA-DR or CD4. Dendritic cell progenitors typically lack expression of co-stimulatory molecules CD40, CD80, and CD86 as well as the activation marker CD83. Dendritic cell progenitors also lack expression of CD3, CD56, and CD20, markers associated with T-cell, NK cell, or B-cell lineages.

“Mature dendritic cell or DC”, as used herein, refers to a mature antigen presenting cell, which is identified by the expression of one or more of the following markers on its cell surface CD54, CD40, CD80, CD86, CD83 and HLA-DR.

“Type 1 dendritic cells or DC1” refer to mature dendritic cells or dendritic cell precursors that express CD11c and CD1b but lack expression of BDCA-2.

“Type 2 dendritic cells or DC2” refer to mature dendritic cells or dendritic cell precursors that lack expression of CD11c and lack expression of CD1b but express high levels of CD123 and BDCA-2.

“Hematopoietic system reconstituting cells” means a population of cells, preferably human, that possess the capability of dividing and producing progeny that include all of the formed cellular elements of the blood. Sources of hematopoietic system reconstituting cells can include bone marrow (both fetal and adult) and peripheral blood mononuclear cells (PBMC).

“Donor” or “donor source” means the animal, preferably human, that is the naturalsource from which cells are originally removed.

“Recipient” means the animal, typically human, into which cells or liposomes are transplanted.

“Allogeneic” means that the recipient is not the natural source from which transplant cells have been removed.

B. Components and Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if a particular liposome is disclosed and discussed and a number of modifications that can be made to a number of molecules including the liposome are discussed, specifically contemplated is each and every combination and permutation of liposomes and the modifications that are possible unless specifically indicated to the contrary. Thus, if a class of liposomes A, B, and C are disclosed as well as a class of antigens D, E, and F, and an example of a combination molecule (that is, for example, an antigen-loaded liposome) A-D is disclosed, then even if each is not individually recited, each combination is individually and collectively contemplated. This means, in this example, that combinations, A-E, A-F, B-D, B-E, B-F, C-D, C-E, and C-F should be considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

C. Dendritic Cells

Dendritic cells (DC) are antigen presenting cells which are important in the initiation of immune responses. Based on in vitro culturing and characterization, DC are thought to represent a family of related cell types, perhaps from a single lineage. Progenitors of DC (DCP) can now be identified in many tissues, such as bone marrow and blood, based on the expression of certain cell surface markers. One type of precursor cell, the Langerhans cell, is found in the epidermis. Work on Langerhans cells established that these immature precursors are capable of antigen uptake, but do not demonstrate a significant ability to stimulate T cells until they differentiate into mature DC.

A variety of markers have been identified that are useful in defining certain cell types of the dendritic cell family. For example, markers expressed on the surface of mature dendritic cells can include CD1a, CD11c, DEC-205 (mouse), CD4, CD33, CD208 (DC-LAMP), CD54, CD11b. HLA-DR, CD40, CD80, CD83, and CD86. Typically, the following markers are not found on the surface of mature dendritic cells: CD20, CD3, CD14, CD16, CD2, CD207 (Langerin), E-cadherin, Birbeck granules, CD56, and CD19. Cell surface markers present on type-I dendritic cell precursors include CD11c, CD13, CD14, CD33, CD34, and HLA-DR. Cell surface markers present on type-2 dendritic cell precursors include CD4, CD45RA, CD34, CD123, HLA-DR, and CD36, while the following markers are not found on the surface of dendritic cell precursors: CD20, CD3, CD16, CD2, CD40, CD80, CD83 and CD86 (see for example Caux et al. Intl. Immunology 1994 6(8): 1177-1185; Steptoe et al. J. Immunology 1997 159:5483-5491; Grouard et al. (J. Exp. Med. 1997 185(6): 1101-1111; O'Doherty et al. Immunology 1994 82: 487-493; Galy et al., J. Immunotherapy 1998 21(2): 132-141; Strunk et al., J. Exp. Med. 1997 185(6): 1131-1136; Olweus et al. Proc. Natl. Acad. Sci. USA 1997 94:12551-12556; Prignano et al., Intl. J. Dermatol. 1998 37:116-119). By the appropriate use of these markers, dendritic cells can be distinguished from dendritic cell progenitors.

In particular, dendritic cells are mature antigen presenting cells, which can be identified by the expression of one or more of the following markers on its cell surface: CD40, CD80, CD86, CD83 and HLA-DR. Further, unless the context indicates otherwise, a dendritic cell progenitor is a “dendritic cell.” Dendritic cell progenitors are hematopoietic cells which can be identified by the expression of one or more of the following markers on its cell surface: Type-1 dendritic cell progenitors—CD11c, CD13, CD14, CD33, CD34, and HLA-DR. Type-2 dendritic cell progenitors—CD4, CD45RA, CD34, CD123, HLA-DR, and CD36.

1. Type 2 Dendritic Cells

Type 2 Dendritic cells or “plasmacytoid” dendritic cells are characterized as hematopoietic cells that can present antigen in the context of both MHC class I and MHC class II. These cells are CD11b−, CD11c−, CD123^{bright}, BDCA-2+, BDCA-4+, HLA-DR+, lineage-(CD3−, CD14−, CD19−, and CD56−). In addition to presenting antigen to T cells type 2 dendritic cells also secrete IL-4 which suppresses IL-12 secretion by T-cells and redirects T-cell immune responses from a T_{H}1 response to T_{H}2. Type 2 dendritic cells are largely responsible for presenting allergens to T cells.

2. Type 1 Dendritic Cells

Type 1 Dendritic cells are characterized as hematopoietic cells that can present antigen in the context of both MHC class I and MHC class II. These cells are CD11b+, CD11c+, CD123^{dim}, BDCA-1+, HLA-DR+, lineage-(CD3−, CD14−, CD19−, and CD56−). In addition to presenting antigen to T cells upon antigen encounter and co-stimulation through CD40-CD40L interactions, type 1 dendritic cells also up regulate IL-12 and secrete IFN-γ which suppresses direct T-cell immune responses towards a T_{H}1 response. Type 1 dendritic cells are largely responsible for presenting bacteria, viruses, and intracellular parasites to T cells.

3. Isolation of Dendritic Cells

Dendritic cells to be incubated with antigens can be isolated using any suitable techniques. Useful techniques involve selection or removal of cells based on the presence of cell surface markers. Many cells, including dendritic cells useful for the disclosed method, carry and lack unique sets of cell surface markers. Useful markers identifying dendritic cells are described above and elsewhere herein. Any one or a combination of these markers can be used in the isolation of dendritic cells. Particularly useful for the disclosed method are the markers CD11c, CD123 and BDCA-2. The presence of CD123 and BDCA-2, and the absence of CD11c identify a particularly useful dendritic cell subset: type 2 dendritic cells (DC2).

Cells can be selected for retention via markers present on the desired cells. Cells can be selected for removal via markers not present on the desired cells. Thus, the cells remaining after cells are removed via a particular cell marker are enriched for cells lacking that cell marker. Both selection and removal of cells using cell markers can be accomplished using, for example, specific binding molecules targeted to the cell marker involved. Useful specific binding molecules for cell separation are antibodies direct to and/or specific for a cell surface marker. The targeted cells can be separated and/or sorted from cells lacking the marker by any of a variety of techniques. In general, such techniques make use of a tag component associated with the specific binding molecule. For example, the tag component can be a fluorescent label, a paramagnetic bead, or column matrix. Fluorescent labels are useful for cell separation based on the well-established technique of fluorescence activated cell sorting (FACS). Likewise, paramagnetic beads and the like and affinity columns derivatized with appropriate specific binding molecules can be used to separate cells.

Examples of fluorescent labels that can be used as tag components include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels that can be used as tag components include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.18, CY5.18, CY7, Dans (1-Dimethyl Amino Naphaline 5 Sulphonic Acid), Dansa (Diamino Naphtyl Sulphonic Acid), Dansyl NH—CH₃, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

D. Antigens

The disclosed methods make use of antigens to affect the immune response to those antigens. For example, both antigens that cause desirable immune responses, such as antigens derived from pathogens (to be used to induce a protective immune response to the pathogen), and antigens that cause undesirable immune responses, such as antigens involved in auto-immune diseases, allergic reactions and graft rejection. Any antigen of interest can be used. Useful antigens include those involved in an immune response and/or disease or condition. Particularly useful antigens are those that can cause an undesirable immune response. Examples include antigens involved in auto-immune disease, allograft rejection, allergic reactions, allergic immune responses. Useful antigens include those involved in auto-immune disease (referred to herein as auto-immune antigens), such as antigens involved in diabetes mellitus, multiple sclerosis, Chron's disease, inflammatory bowel disease, rheumatoid arthritis, thyroiditis, vitiligo, and/or systemic lupus erythematosis. Useful antigens also include those involved in allograft rejection, such as graft rejection of hematopoietic stem cell transplants.

Particularly useful antigens include MHC, QA, HIV gag, pol, and env (DNA or protein), rheumatoid factor, ICA 89, peripherin, carboxypeptidase H, glutamic acid decarboxylase (GAD), AHNAK, mylein basic protein, retinal S antigen, galactomannoprotein, neuraminidase, influenza matrix protein M1, CMV env proteins pp60, ds DNA, thyroglobulin, insulin, pancreatic islet beta cell antigens, MART-1 (melanoma protein), tyrosinase-related protein 2 (TRP2), melanoma cell lysate, MAGE-A3, allogeneic cell lysate, HLA antigens (protein or DNA), CD31 (protein or DNA), aspergillus chitin, pancreatic carcinoma cell line Panc-1 lysate, ErbB-2/neu, human epithelial cell mucin (Muc-1), Ha-ras oncogene product, p53, carcino-embryonic antigen (CEA), raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), prostate specific membrane antigen (PSMA), HPV-16, MUM, alpha-fetoprotein (AFP), CO17-1A, GA733, gp72, p53, ras oncogene product, HPV E7, Wilm's tumor antigen-1, telomerase, melanoma gangliosides, malignant B cell antigen receptor, malignant B cell immunoglobulin idiotype, variable region of an immunoglobulin, hypervariable region or complementarity determining region (CDR) of a variable region of an immunoglobulin, malignant T cell receptor (TCR), variable region of a TCR, and hypervariable region of a TCR.

For induction of an immune response against cancer or tumor cells, the antigens can be tumor antigens. Tumor antigens for use in the disclosed method can be any tumor antigen now known or later identified as a tumor antigen. The appropriate tumor antigen used depends on the tumor type being treated. For example, the tumor antigen can be, but is not limited to human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, gp100/pmel17, GD2, GD3, GM2, TF, sTn, tyrosinase-related protein 2 (TRP2), melanoma cell lysate, MAGE-A3, MAGE-1, MAGE-3, BAGE, GAGE, tyrosinase, gp75, Melan-A/Mart-1, gp100, HER2/neu, ErbB-2/neu, pancreatic carcinoma cell line Panc-1 lysate, EBV-LMP 1 & 2, HPV-F4, 6, 7, prostate-specific antigen (PSA), prostate specific membrane antigen (PSMA), HPV-16, MUM, alpha-fetoprotein (AFP), CO17-1A, GA733, gp72, p53, the ras oncogene product, HPV E7, Wilm's tumor antigen-1, telomerase, and melanoma gangliosides, as well as any other tumor antigens now known or identified in the future. Tumor antigens can be obtained following known procedures or are commercially available.

Additionally, the tumor antigen of the present invention can be an antibody which can be produced by a B cell tumor (e.g., B cell lymphoma; B cell leukemia; myeloma) or the tumor antigen can be a fragment of such an antibody, which contains an epitope of the idiotype of the antibody. The epitope fragment can comprise as few as nine amino acids. For example, the tumor antigen of this invention can be a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR.

Where the object of treatment is graft rejection, graft versus host disease, or auto-immune disease, the antigens can be prepared using cells from the source of the antigen(s) involved. For example, cells from the source of transplant cells can be used to obtain relevant antigens. As will be appreciated, the source cells contain antigens that are involved in graft rejection. Cells from the individual to be treated can be used to obtain relevant antigens in the case of auto-immune diseases. In general, whole cell preparations can be used. The cells can irradiated to kill the cells and the resulting treated cell mixture can be used as the antigens for incubation with dendritic cells of loading into liposomes targeted to dendritic cells. As a specific example, allo-antigen can be prepared by irradiating peripheral blood mononuclear cells from a third party donor to 25 Gy. Use of antigens prepared in this way is described in the Example.

Useful antigens for altering immune responses in the context of transplants include MHC and QA antigens. Useful antigens for altering immune responses to influenza include neuraminidase, and influenza matrix protein M1. Useful antigens for altering immune responses to CMV include env proteins pp60. Useful antigens for altering immune responses in lupus include ds DNA and AHNAK. Useful antigens for altering immune responses in thyroditis include thyroglobulin. Useful antigens for altering immune responses in diabetes include insulin and pancreatic islet beta cell antigens. Useful antigens for altering immune responses to allografts include allogeneic cell lysate, HLA antigens (protein or DNA), and CD31 (protein or DNA). Useful antigens for altering auto-immune responses include rheumatoid factor, ICA 89, peripherin, carboxypeptidase H, and glutamic acid decarboxylase (GAD). Useful antigens for altering immune responses to fungal infections include aspergillus chitin and galactomannoprotein. Useful antigens for altering immune responses to pancreatic cancer include pancreatic carcinoma cell line Panc-1 lysate. Useful antigens for altering immune responses to prostate cancer include prostate specific antigen. Useful antigens for altering immune responses to colon cancer include carcioembryonic antigen. Useful antigens for altering immune responses to breast, renal, and/or lung cancer include ErbB-2/neu. Useful antigens for altering immune responses to HIV include HIV gag, pol, and env (DNA or protein).

E. Liposomes

The disclosed liposomes comprise liposomes comprising one or more antigens and one or more surface-bound molecules. The antigens are antigens of interest, such as antigens involved in undesirable immune responses. Liposomes comprising antigens are referred to herein as antigen-loaded liposomes. The surface-bound molecules are molecules that target the liposomes to dendritic cells. Liposomes containing surface-bound molecules are referred to herein as targeted liposomes. For convenience, the term liposome is used herein to refer to the base liposome, as well as to antigen-loaded and targeted liposomes. Generally, the context of the term indicates what is meant. Surface-bound molecules generally comprise a component that can bind to and/or interact with targeted cells. In the context of the disclosed method, the targeted cells are dendritic cells. Useful components that can bind to and/or interact with targeted cells are specific binding molecules. In the context of the disclosed method, the specific binding molecule is generally interacts with and/or is specific for a cell surface marker. Particularly useful targets for surface-bound molecules are CD11c, BDCA-1, or both, for targeting mature type 1 dendritic cells, and CD123, BDCA-2, BDCA-4, or a combination, for targeting type 2 dendritic cells.

Liposomes are artificial structures primarily composed of phospholipid bilayers. Cholesterol and fatty acids may also be included in the bilayer construction. Liposomes may be loaded with compounds of interest, such as the disclosed antigens, and coated on the outer surface with surface-bound molecules, such as specific binding molecules. Liposomes, preferably unilamellar vesicles, can be made using established procedures that result in the loading of the interior compartment with antigens. The liposomes can also be associated with a surface-bound molecules, such as a specific binding molecule. The association may be direct or indirect. An example of a direct association is a liposome containing covalently bound antibodies on the surface of the phospholipid bilayer. An example of indirect association is a liposome containing covalently bound anti-antibody antibodies that can bind targeted antibodies.

Liposome-forming compounds are generally well known as are the methods of their preparation. For example, any number of phospholipids or lipid compounds may be used to form the vesicle walls. Representative of such wall forming compounds are; phosphatidylcholine (hereinafter referred to as “PC”), both naturally occurring and synthetically prepared, phosphatidic acid (hereinafter referred to as “PA”), lysophosphatidylcholine, phosphatidylserine (hereinafter referred to as “PS”), phosphatidylethanolamine (hereinafter referred to as “PE”), sphingolipids, phosphatidyglycerol (hereinafter referred to as “PG”), spingomyelin, cardiolipin, glycolipids, gangliosides, cerebrosides and the like used either singularly or intermixed such as in soybean phospholipids. In addition, other lipids such as steroids, cholesterol, aliphatic amines such as long chain aliphatic amines and carboxylic acids, long chain sulfates and phosphates, dicetyl phosphate, butylated hydroxytoluene, tocophenol, retinol, and isoprenoid compounds may be intermixed with the phospholipid components to confer certain desired and known properties on the formed vesicles. In addition, synthetic phospholipids containing either altered aliphatic portions such as hydroxyl groups, branched carbon chains, cycloderivatives, aromatic derivatives, ethers, amides, polyunsaturated derivatives, halogenated derivatives or altered hydrophillic portions containing carbohydrate, glycol, phosphate, phosphonate, quaternary amine, sulfate, sulfonate, carboxy, amine, sulfhydryl, imidazole groups and combinations of such groups can be either substituted or intermixed with the above mentioned phospholipids and used in the disclosed method. It will be appreciated from the above that the chemical composition of the lipid component of the vesicles prepared by the method of the invention may be varied greatly without appreciable diminution of percentage capture although the size of the vesicle may be affected by the lipid composition. A convenient mixture we have used extensively and which is representative of lipid mixtures advantageously used in the method of the invention is composed of PS and PC, or PG and PC as identified above (advantageously at a 1:4 molar ratio in each instance). The PC, PG, PA and PE, may be derived from purified egg yolk. Saturated synthetic PC and PG, such as dipalmitoyl may also be used. Other amphipathic lipids that may be used, advantageously also at 1:4 molar ratios with PC, are gangliosides, globosides, fatty acids, stearylamine, long chain alcohols, and the like.

The production and use of liposomes, including description of liposomes having useful properties and specialized components, are known. For example, various liposomes, their components and their method of production and use are described in U.S. Pat. No. 6,296,870, U.S. Pat. No. 6,241,999, U.S. Pat. No. 6,214,388, U.S. Pat. No. 5,354,853, U.S. Pat. No. 4,900,556, U.S. Pat. No. 4,708,861, U.S. Pat. No. 4,235,871, and U.S. Pat. No. 4,224,179, all of which are incorporated herein by reference for their description of he above subject matter. The class of liposomes described in U.S. Pat. No. 6,214,388, which are designed to maximize internalization of liposomes by cells having cell surface markers of interest, are particularly useful in the disclosed method.

1. Modifying Liposomes

Disclosed is a method of modifying liposomes comprising loading liposomes with one or more antigens. The liposomes can also be modified by adding surface-bound molecules to the surface of the liposomes. The surface-bound molecules can target the liposomes to cells, such as dendritic cells. Techniques for loading liposomes and for producing liposome having surface-bound molecules are known. For example, liposomes can be loaded with antigens by forming the liposomes in the presence of the antigens (see, for example, liposome patents described above). Liposomes having surface-bound molecules can be produced, for example, by using lipids to which the surface-bound molecules are couple or otherwise attached. It is useful to couple surface-bound molecules at or near a polar or charged portion of the lipids. This should allow the surface-bound molecules to be exposed on the surface of the liposomes.

F. Specific Binding Molecules

A specific binding molecule is a molecule that interacts specifically with a particular molecule or moiety. In the context of the disclosed method, the molecule or moiety that interacts specifically with a specific binding molecule is generally a cell surface marker. Antibodies, either member of a receptor/ligand pair, synthetic polyamides (Dervan and Burli, Sequence-specific DNA recognition by polyamides. Curr Opin Chem Biol, 3(6):688-93 (1999); Wemmer and Dervan, Targeting the minor groove of DNA. Curr Opin Struct Biol, 7(3):355-61 (1997)), and other molecules with specific binding affinities are examples of specific binding molecules. Specific binding molecules are generally used to isolate useful dendritic cells and/or to target liposomes to dendritic cells as described elsewhere herein.

A specific binding molecule that interacts specifically with a particular analyte is said to be specific for that analyte. For example, where the specific binding molecule is an antibody that associates with a particular antigen, the specific binding molecule is said to be specific for that antigen. The antigen is the analyte. A reporter molecule containing the specific binding molecule can also be referred to as being specific for a particular analyte. Specific binding molecules preferably are antibodies, ligands, binding proteins, receptor proteins, haptens, aptamers, carbohydrates, synthetic polyamides, peptide nucleic acids, or oligonucleotides. Preferred binding proteins are DNA binding proteins. Preferred DNA binding proteins are zinc finger motifs, leucine zipper motifs, helix-turn-helix motifs. These motifs can be combined in the same specific binding molecule.

1. Antibodies

Antibodies useful as specific binding molecules in the disclosed method are generally specific for cell surface markers. Numerous antibodies specific for various cell surface markers are known, and many are commercially available. Such antibodies can be used in the disclosed method and/or to produce components used in the disclosed method (such as for use as specific binding molecules). Useful antibodies can also be produced for use in the disclosed method. Techniques for antibody production are known, some of which are described below. Such techniques can be used to produce antibodies for use in the disclosed method.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with cell surface markers (or other antibody target), as described herein. The antibodies can be tested for their desired activity using the in vitro assays described herein, or by analogous methods.

The term “monoclonal antibody” as used herein refers to an antibody obtained from a substantially homogeneous population of antibodies, that is, the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

Monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (for example, by using oligonucleotide probes that are capable of binding specifically to genes encoding the heavy and light chains of murine antibodies). Libraries of antibodies or active antibody fragments can also be generated and screened using phage display techniques, for example, as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992). As used herein, the term “antibody” or “antibodies” can also refer to a human antibody and/or a humanized antibody.

G. Tag Components

Tag components are molecules that can be associated with specific bindingmolecules and used to separate or sort cells. Useful tag components include fluorescent molecules and sortable components. Fluorescent molecules are particularly useful for fluorescence activated cell sorting (FACS). Sortable components include molecules and compositions that can be selectively bound or that can selectively interact with other molecules and compounds. Examples include beads or particles, such as paramagnetic bead, that can be separated based on a property of the bead. Thus, for example, cells associated with paramagnetic bead or particle can be selected or separated by magnetic sorting. Specific binding molecules are a useful form of tag component. Cells associated with such a specific binding molecule can be separated by associating the specific binding molecule with its binding partner (that is the molecule or moiety with which the specific binding molecule interacts specifically). Thus, for example, cells with which a specific binding molecule (as tag component) is associated can be selected or separated by passing the cells over a column derivatized with the binding partner of the specific binding molecule.

H. Incubation of Dendritic Cells With Antigens

Some forms of the disclosed methods involve or make use of dendritic cells that have been incubated with or exposed to one or more antigens. In particular, the disclosed method can use type 2 dendritic cells or CD11c−, CD123^{bright}, BDCA-2+ dendritic cells that have been incubated with one or more antigens. Administration of such dendritic cells to an individual can induce immunological tolerance to the antigens. Alternatively, the disclosed method can use immature type 1 dendritic cells or CD11c−, CD123^{dim}, BDCA-2− dendritic cells that have been incubated with one or more antigens. Administration of such type 1 dendritic cells to an individual can enhance immune responses to, for example, tumor antigens and antigens associated with infectious diseases.

Dendritic cells are generally incubated with antigens ex vivo. In this context, ex vivo refers to treatment of the cells outside of the body from which they are derived or isolated. If ex vivo methods are employed, the cells can be maintained outside the body according to standard protocols well known in the art. The antigens can be incubated with the cells under any suitable conditions. Useful conditions include conditions useful for maintenance of the cells outside the body.

As a specific example, preparations of antigens from irradiated cells can be incubated with dendritic cells. For example, 20,000 irradiated cells can be added to isolated dendritic cells and the cells incubated for 24 and 72 hours in a 5% CO₂ atmosphere at 37° C. TNF can then be added (at, for example, 10 ng/ml) to mature and activate dendritic cell progenitors. Use of these incubation conditions is described in the Example.

I. Administration of Dendritic Cells

Dendritic cells exposed to antigen can be administered to individuals and animals using any suitable technique. In general, The treated cells can be infused per standard methods for the cell type. Standard methods are known for infusion of dendritic cells into a subject. In general, the disclosed dendritic cells can be administered by infusion, such as by intravenous infusion. A useful method of intraveneous infusion is to insert a indwelling catherter into a vein and attach to the catheter a flexible tubing, usually composed of plastic, and attach to the tubing a reservoir containing the substance to be infused. The reservoir may be a bag or syringe containing a liquid solution. The solution to be infused is transferred from the reservoir bag through the flexible tubing and indwelling catherter into the venous circulation of the recipient. The force to affect the transfer of the solution into the venous circulation may be gravity or compression of the bag or syringe. However, the dendritic cells may also be administered orally, parenterally (for example, intravenously), by intramuscular injection, by intraperitoneal injection, transdermally, sub-cutaneously, topically or the like. As used herein, “intravenous infusion” means delivery of the cells into a vein of the individual. Administration of the dendritic cells by inhalant can be through the nose or mouth via delivery by a spraying or droplet mechanism.

J. Administration of Liposomes

Liposomes can be administered to individuals and animals using any suitable technique. In general, the disclosed liposomes can be administered by infusion, such as by intravenous infusion. This is useful because the targeted dendritic cells are found in the blood. However, the liposomes may be administered orally, parenterally (for example, intravenously), by intramuscular injection, by intraperitoneal injection, transdermally, sub-cutaneously, topically or the like. As used herein, “intravenous infusion” means delivery of the liposomes into a vein of the individual. Administration of the liposomes by inhalant can be through the nose or mouth via delivery by a spraying or droplet mechanism. Delivery can also be directly to any area of the respiratory system (for example, lungs) via intubation. The exact amount of the liposomes required will vary from subject to subject, depending on the species, age, weight and general condition of the subject, the mode of administration and the like. Thus, it is not possible to specify an exact amount for every liposome. However, an appropriate amount can be determined by one of ordinary skill in the art using only routine experimentation given the teachings herein.

K. Immune Responses

An immune response refers to one or more effects produced by the immune system in reaction to one or more antigens. This includes, for example, immune system reactions to combinations of antigens; combinations of one or more antigens with other, non-antigenic components; and antigens in or on cells, viruses, liposomes or other compositions. As used herein, immune response refers to the anti-antigen function of the immune system and not to effects on and changes in the immune system that reduce the anti-antigen function of the immune system. For example, stimulation of neutralizing antibodies in response to exposure of an immune system to an antigen is an immune response, while induction of immunological tolerance is not an immune response as that term is used herein. It should be understood that this is the case even though immunological tolerance represents an effect produced by the immune system in reaction to one or more antigens.

Adaptive immune response can largely be differentiated into two components humoral and cell-mediated. This division is based on whether the immune response is dominated by antibodies produced by plasma cells (a B cell lineage cell) or cytotoxic T cells. However, rarely are immune responses strictly the domain of either one of the components. Predominantly, the immune response is a combination of the two arms of the immune response having both T cell and B cell components.

The dominance of either component is largely the result of regulatory CD4+ T cells, and the interaction of these cells with antigen in the context of dendritic cells. CD4 T cells can produce a myriad of functions such as cytokine release and direct cytotoxicity. It is these functions that determines whether the cell-mediated or humoral responses will dominate. Typically, CD4 T cells that stimulate a cell-mediate response are referred to as inflammatory CD4 T cells or T_{H}1 cells. A T_{H}1 response is characterized by IFN-γ, TNF, and IL 12 production from CD8 and CD4 T cells, IgG2a antibody production, and the generation of cytotoxic T cells. CD4 cells that induce a more humoral response are termed T_{H}2. T_{H}2 responses result in IL-4, IL5, and IL10 secretion and IgG1, IgA and IgE antibody production. The determination as to whether a CD4 cell will become a T_{H}1 or T_{H}2 cell is largely a factor of the environment that surround the cell at the time of activation. The environmental factors that control this are cytokines and the type of “non-professional” antigen-presenting cells or “professional” antigen-presenting cells (dendritic cells) that presents the antigen. Secreted IL-12 and IFN-γ in the presence of an uncommitted T cell can promote a T_{H}1 response. Similarly, secreted IL-4 in the presence of an uncommitted T cell can promote a T_{H}2 response. Additionally, these cytokines have the effect of suppressing the opposite response.

The environment associated with the determination of the commitment to a T_{H}1 or T_{H}2 pathway also involves the cells that present antigen to CD4 T cells. CD4 T cells recognize peptide antigen in the context of MHC class II molecules presented by antigen presenting cells. These cells may be macrophage, mast cells, dendritic cells, or B cells. Depending on the cell type that presents the antigen the CD4 cell may be committed to either pathway. CD4 T cells activated by macrophage or type 1 dendritic cells are directed to induce a cell-mediated immune response. CD4 T cells activated by type 2 dendritic cells and mast cells are directed to induce a T_{H}2 immune response.

1. Reduction of Immune Responses

Some embodiments of the disclosed methods result in reduction of an immune response. As used herein, reduction of an immune response refers to a lessening of one or more effects produced by the immune system in reaction to an antigen. Such reduction includes both reduction of an actual immune response or a reduction in an immune response that would otherwise have been expected in the absence of the reducing treatment. Reduction can include, but does not require, lessening of the immune response to an undetectable level. As used herein, suppression of an immune response refers to a lessening of one or more effects produced by the immune system in reaction to an antigen. Such suppression includes both lessening of an actual immune response or a lessening of an immune response that would otherwise have been expected in the absence of the suppressing treatment. Suppression can include, but does not require, lessening of the immune response to an undetectable level.

2. Enhancement of Immune Responses

Some embodiments of the disclosed methods result in enhancement of an immune response. As used herein, enhancement of an immune response refers to an increase of one or more effects produced by the immune system in reaction to an antigen. Such enhancement includes both enhancement of an actual immune response or an enhancement in an immune response that would otherwise not have been expected in the absence of the enhancing treatment. As used herein, generation of an immune response refers to the creation of one or more effects produced by the immune system (to a detectable level) in reaction to an antigen. Such generation includes creation of an immune response that would otherwise not have been expected in the absence of the generating treatment.

L. Immunological Tolerance

Some forms of the disclosed methods result in immunological tolerance to one or more antigens. As used herein, immunological tolerance refers to a physiological state in which the immune system does not react destructively against antigens. In effect, immunological tolerance is a lack of an immune response to an antigen. Mechanistically, immunological tolerance generally is the result of either deletion of reactive cells during development, such as “central tolerance” that occurs during deletion of auto-reactive T-cells during intrathymic development, or peripheral tolerance, that may occur during encounter of immune cells with antigen at peripheral lymphoid tissues in a manner that generates an anergis or unresponsive state.

Immunological tolerance generally involves interruption of one or more steps involved in generation of an immune response. For example, individuals generally have immunological tolerance to self-antigens. This tolerance is a result of negative selection that takes place during T and B cell development in the thymus and bone marrow respectively. For T cells, this process involves the clonal deletion of T cells that recognize self-antigens. The deletion takes place in the thymus. Here CD4+CD8+ T cells are transitioning to either CD4−CD8+ or CD4+CD8− T cells. At this point in their development, the T cells are positively selected based on exposure and the subsequent ability to bind self-MHC. T cells that do not respond to the stimuli undergo activation induced cell death (AICD) and are deleted from the T cell repertoire, as being unable to recognize self-MHC would prevent the recognition of antigen since a TCR will only recognize antigen in the context of MHC. Those T cells that recognize self-MHC (that is, bind above a certain threshold) finish the differentiation to CD4−CD8+ or CD4+CD8− T cells. The surviving T cells are then negatively selected if they become activated by self-antigen being presented on self-MHC. This negative selection insures that the immune system won't respond to self and the responding T cells are deleted. Interestingly, foreign antigen present in the organism at the time of negative selection will also be seen as self. An example of this is the creation of Lymphocytic choriomeningitis virus (LCMV) carrier mice via neonatal infection. Adult mice infected with LCMV have a profound T cell response which can lead to meningitis due to immune pathology. Though a persistent infection can ensue, mice not dying via immune pathology will clear the infection. However, neonatal mice infected with LCMV will not generate an immune response and will become lifelong carriers of the virus showing no ill effects of infection or immune pathology. Similarly, mice receiving a non-lethal dose of irradiation will lose all T cells. Exposure to a foreign antigen prior to the reconstitution of the T cell population will result in animals that are tolerant to the foreign antigen.

Though the mechanism of central tolerance through clonal deletion insures that the majority of T cells do not recognize self, there are tissue-specific antigens in the periphery that will not be presented to developing T cells in the thymus. Therefore, it is still possible to have T cells that react to self. However, the activation of a T cell is a two step process (1) the T cell must recognize antigen in the context of self-MHC and (2) the T cell must receive co-stimulatory signals in the form of CTLA-4-CD80/CD86, 41BB-41BBL, CD40-CD40L or CD28-B7.1/B7.2 interactions. Without both signals, a T cell will not become activated. In the situation of peripheral antigens, T cells specific for these self-antigens do not receive a co-stimulatory signal and therefore become anergic.

In contrast to T cells, B cells do not recognize antigen in the context of MHC and therefore do not require a positive selection step. However, immature B cells do undergo negative selection within the bone marrow. Here, B cells with immunoglobulin (BCR) that recognizes self-antigens are clonally deleted. This was shown experimentally in mice transgenic for transmembrane bound hen-egg lysozyme (HEL) and transgenic for B cells specific for HEL. In these experiments, immature B cells specific for HEL can be detected, but no HEL specific mature B cells exist as they are deleted from the repertoire.

Despite the deletion of self-specific cells in the bone marrow, there is still potential for mature B cells to exist that recognize soluble self-antigen or antigen only expressed in certain peripheral tissue. In the latter situation, B cells that recognize antigen expressed in peripheral tissue are deleted. However, B cells that recognize soluble antigen are not deleted. Instead these B cells internalize their antigen receptor and lose functionality. This loss of function is called anergy and results in a lack of response to an antigen despite the presence of specific cells.

M. Pharmaceutically Acceptable Carriers

The disclosed cells and liposomes can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. Particularly useful pharmaceutical carriers are physiological solutions. In the case of the disclosed cells, media and solutions in which the cells are store, grown, maintained or. incubated are particularly useful as pharmaceutical carriers.

Pharmaceutical compositions may include carriers, thickeners, diluents, buffers, preservatives, surface active agents and the like in addition to the cells or liposomes. Pharmaceutical compositions may also include one or more active ingredients such as antimicrobial agents, anti-inflammatory agents, anesthetics, and the like.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLE

A. The Effect of DC2p Depletion on the Ability of the Cells in an HPC Product to Generate an Allo-Immune Response.

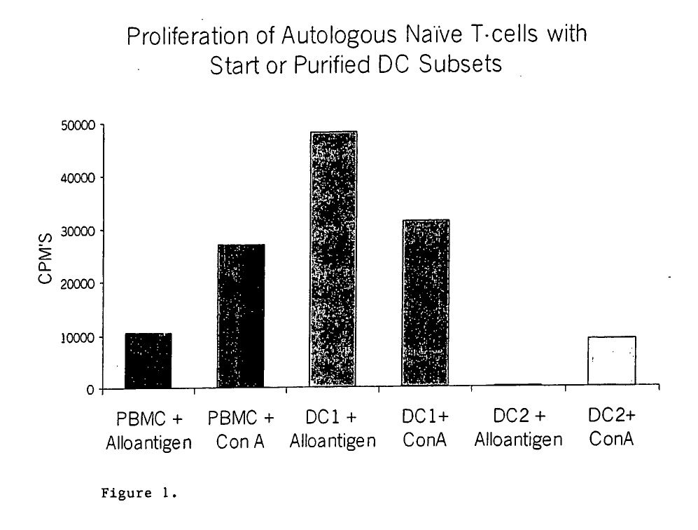

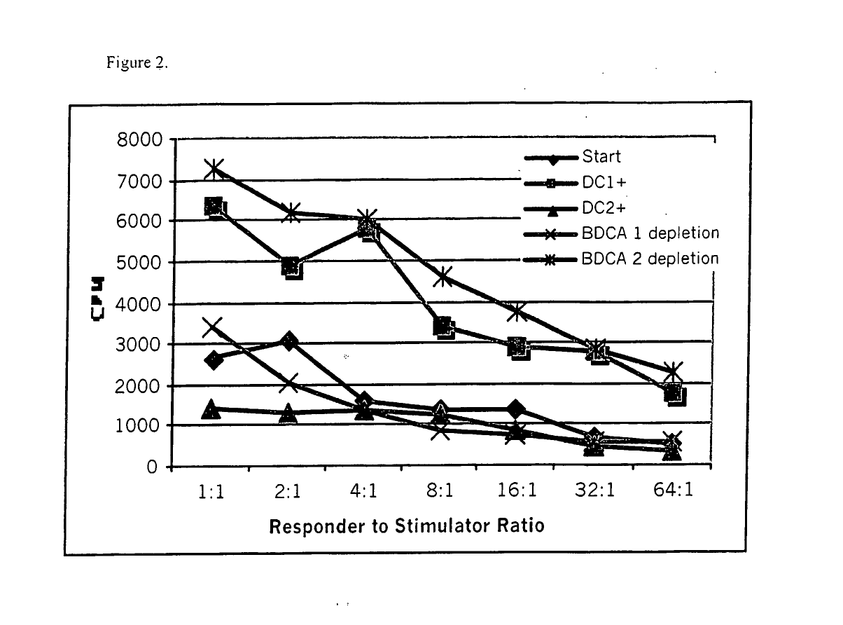

The mononuclear cell fraction of the blood from a normal donor was collected by apheresis. Naïve CD45RA+ 62L+ T-cells were purified by FACS and frozen. The HPC product was depleted of DC2p using the BDCA2 antibody and the MACS immuno-affinity columns or purified DC1p and FACS isolated DC2p populations. The ability of the DC2p depleted HPC cells and the purified DCp2 and DC1p populations to generate cellular immune responses to allo-antigen was determined by adding allo-antigen to the DCp cells, maturing the DCp by the addition of IL3, GM-CSF, and TNF, then adding the previously collected naïve T-cells and measuring their proliferation in response to the allo-antigen (FIGS. 1 and 2). The proliferative response of autologous T-cells to allo-antigen presented by purified DC2p was inhibited, while T-cells showed marked proliferation in response to allo-antigen presented by purified DC1p. Of note, T-cells had a strong proliferative response to the HPC graft that was depleted of the inhibitory DC2p population (stars, FIG. 2).

Procedural Details.

Peripheral blood mononuclear cells were obtained form a normal donor by apheresis. Naïve CD3+, CD45RA+, CD62L+ naïve T-cells were isolated following staining with fluorescently labeled anti-CD3, anti-CD45RA, and anti 62L and high speed fluorescent activated cell sorting. DC1 (CD11c+, CD123dim, lineage-) and DC2 (CD11c−, CD123+, lineage-) populations were enriched from the apheresis product using immuno-affinity magnetic purification using antibodies to DC1 (BDCA-1) and DC2 (BDCA-2) then purified to >95% homogeneity by high speed cell sorting after staining with monoclonal antibodies to CD123, CD11c and a cocktail of lineage markers (CD3, CD14, CD19, and CDF56). Defined numbers of mononuclear cells (start fraction), purified DC1, purified DC2, or mononuclear cells depleted of either DC1 or DC2 by immuno-magnetic affinity column chromatography were added to tissue culture wells in a 96 well plate and cultured with 50 ng/ml GM-CSF and 50 ng/ml IL3 (start, DC2, and DC1 depleted) or 50 ng/ml GM-CSF and 50 ng/ml IL4 (DC1, DC2 depleted). Allo-antigen was prepared by irradiating peripheral blood mononuclear cells from a third party donor to 25 Gy then adding 20,000 cells to each well containing stimulator cells. After 24 and 72 hours of culture in a 5% CO₂ atmosphere at 37° C., TNF was added at 10 ng/ml to mature and activate DC progenitors. 50,000 autologous naïve T-cells were added to each well and tritiated thymidine incorporation was measured after an additional 96 hours of culture. FIGS. 1 and 2 show the results.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

Those skilled in the art will recognize, or be able to ascertain using no more than routine experimentation, many equivalents to the specific embodiments of the invention described herein. Such equivalents are intended to be encompassed by the following claims.

1-27. (canceled)

28. A method of altering an immune response comprising
administering to the individual liposomes, wherein the liposomes comprise one or more antigens, wherein the liposomes are modified with surface-bound molecules that target the liposomes to type 2 dendritic cells or mature type 1 dendritic cells.

29. The method of claim 28, wherein the antigens are carbohydrates.

30. The method of claim 28, wherein the antigens are nucleic acids.

31. The method of claim 28, wherein the antigens are peptides.

32. The method of claim 28, wherein the antigens are lipids.

33. The method of claim 28, wherein the antigens are a combination of two or more of the following: carbohydrates, nucleic acids, peptides, or lipids.

34. The method of claim 28, wherein the antigens are MHC, QA, HIV gag, pol, and env (DNA or protein), rheumatoid factor, ICA 89, peripherin, carboxypeptidase H, glutamic acid decarboxylase (GAD), AHNAK, mylein basic protein, retinal S antigen, galactomannoprotein, neuraminidase, influenza matrix protein M1, CMV env proteins pp60, ds DNA, thyroglobulin, insulin, pancreatic islet beta cell antigens, MART-1 (melanoma protein), tyrosinase-related protein 2 (TRP2), melanoma cell lysate, MAGE-A3, allogeneic cell lysate, HLA antigens (protein or DNA), CD31 (protein or DNA), aspergillus chitin, pancreatic carcinoma cell line Panc-1 lysate, ErbB-2/neu, human epithelial cell mucin (Muc-1), Ha-ras oncogene product, p53, carcino-embryonic antigen (CEA), raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), prostate specific membrane antigen (PSMA), HPV-16, MUM, alpha-fetoprotein (AFP), CO17-IA, GA733, gp72, p53, ras oncogene product, HPV E7, Wilm's tumor antigen-1, telomerase, melanoma gangliosides, malignant B cell antigen receptor, malignant B cell immunoglobulin idiotype, variable region of an immunoglobulin, hypervariable region or complementarity determining region (CDR) of a variable region of an immunoglobulin, malignant T cell receptor (TCR), variable region of a TCR, hypervariable region of a TCR, or a combination.

35. The method of claim 28, wherein the surface-bound molecule is a monoclonal antibody.

36. The method of claim 28, wherein the surface-bound molecule is a peptide high-affinity ligand.

37. The method of claim 28, wherein the surface-bound molecule is specific for CD11c+, BDCA-1, or both, wherein mature type 1 dendritic cells are targeted.

38. The method of claim 37, wherein the liposomes enhance anti-tumor immune responses.

39. The method of claim 37, wherein the liposomes enhance anti-cancer immune responses.

40. The method of claim 28, wherein the surface-bound molecule is specific for CD123, BDCA-2, BDCA-4, or a combination, wherein type 2 dendritic cells are targeted.

41. The method of claim 40, wherein the liposomes reduce an auto-immune response.

42. The method of claim 41, wherein the auto-immune response is involved in diabetes mellitus, multiple sclerosis, Chron's disease, inflammatory bowel disease, rheumatoid arthritis, thyroiditis, vitiligo, and systemic lupus erythematosis.

43. The method of claim 40, wherein the liposomes reduce an allergic immune response.

44. The method of claim 43, wherein the allergic immune response is involved in asthma, gluten allergy, and atopic dermatitis.

45. The method of claim 40, wherein the liposomes reduce graft rejection immune response.

46. The method of claim 45, wherein the individual is an allograft recipient.

47. The method of claim 45, wherein the graft rejection immune response comprises complications associated with graft rejection.

48. The method of claim 45, wherein the graft rejection immune response comprises graft rejection.

49. The method of claim 40, wherein the individual is a hematopoietic stem cell recipient.

50. The method of claim 40, wherein the liposomes reduce graft verses host immune response.

51. The method of claim 40, wherein the antigens are auto-immune antigens.

52. The method of claim 51, wherein the auto-immune antigens are involved in diabetes mellitus, multiple sclerosis, Chron's disease, inflammatory bowel disease, rheumatoid arthritis, thyroiditis, vitiligo, or systemic lupus erythematosis.

53. The method of claim 28, wherein the antigens are antigens involved in allograft rejection, wherein the liposomes reduce the allograft rejection.

54. The method of claim 28, wherein the antigens are antigens involved in graft rejection of hematopoietic stem cell transplants, wherein the liposomes reduce the graft rejection.

55. The method of claim 28, wherein the antigens are antigens involved in graft verses host disease.

56. A method of reducing an immune response comprising
administering to the individual liposomes, wherein the liposomes comprise one or more antigens, wherein the liposomes are modified with surface-bound molecules that target the liposomes to type 2 dendritic cells.

57. The method of claim 28, wherein the liposomes enhance an immune response, wherein the liposomes are targeted to immature type 1 dendritic cells.

58. The method of claim 28, wherein the liposomes reduce an immune response, wherein the liposomes are targeted to type 2 dendritic cells.

59. The method of claim 58, wherein the type 2 dendritic cells are immature type 2 dendritic cells.

60. A method of enhancing an immune response comprising
administering to the individual liposomes, wherein the liposomes comprise one or more antigens, wherein the liposomes are modified with surface-bound molecules that target the liposomes to mature type 1 dendritic cells.

61. A method of modifying liposomes comprising
packaging one or more antigens into a lipopsome, wherein the liposome is modified with surface-bound molecules that target the liposome to type 2 dendritic cells or mature type 1 dendritic cells.
